(19) United States
(12) Patent Application Publication
(10) Pub. No.: US 2017/0354929 A1
(43) Pub. Date: Dec. 14, 2017

(54) Multiple Location Water Conductivity Measuring Device Applied within a Membrane Vessel

(71) Applicant: Wesley Charles Byrne, Orono, MN (US)

(72) Inventor: Wesley Charles Byrne, Orono, MN (US)

(21) Appl. No.: 15/613,174

(22) Filed: Jun. 3, 2017

Related U.S. Application Data
(60) Provisional application No. 62/347,392, filed on Jun. 8, 2016.

(51) Int. Cl. B01D 61/10; B01D 65/10; G01N 33/18; G01N 27/07; B01D 63/10; B01D 61/02

(52) U.S. Cl. CPC B01D 61/10; B01D 63/10; B01D 61/025; G01N 33/18; G01N 27/07; B01D 61/027; B01D 65/104

(57) ABSTRACT

This invention uses multiple pairs of electrodes acting as electrical conductivity sensors that are secured at specific locations within spiral wound membrane elements and their interconnecting components of a reverse osmosis or nanofiltration pressure vessel. Each electrode pair might be attached to a wire cord to be inserted through and sealed against a vessel end cap into the permeate carrier tubes and interconnecting components of the membrane elements, or each electrode pair might be attached to a battery and a wireless transmitting device. Conductivity measurements from the sensors would be communicated to a microprocessor, which would evaluate each permeate conductivity measurement relative to other permeate conductivity measurements, as well as relative to derived or measured conductivities in the saline water in calculating a percent salt passage value specific to the location of each permeate sensor.

### CROSS REFERENCE TO RELATED APPLICATIONS

This application is a conversion from non-provisional application 62347392 filed Jun. 8, 2016 entitled “CondCord™ Membrane Performance Monitor, a diagnostic tool for monitoring the electrical conductance of the produced purified water at numerous locations from within the adjacent permeate carrier tubes of the membrane elements installed within a membrane housing/pressure vessel of a reverse osmosis or nanofiltration membrane system.”

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND OF THE INVENTION

This invention relates to the automation of methods used to calculate the percent passage of dissolved salts through a spiral wound reverse osmosis (RO) or nanofiltration (NF) membrane at specific locations within the membrane elements' permeate carrier tubes and interconnecting components. By applying this invention in measuring the electrical conductance of the water at multiple locations within a membrane pressure vessel without altering the permeate flow stream, its microprocessor compares conductivity values in such a manner as to derive accurate values for the percent passage of dissolved salt at each of its permeate stream measurement locations.

A reverse osmosis (RO) or nanofiltration (NF) membrane system applies water pressure to remove dissolved salt from a water source using spiral-wound membrane elements installed within a pressurized vessel. The pressure vessels that may contain up to eight 40-inch long cylindrical membrane elements installed end-to-end. The spiral-wound membrane element uses a permeate carrier tube situated lengthwise down its center to collect the water that permeates through the membrane sheets within the element. The permeate carrier tube also serves to transport the permeated water (aka, permeate) produced by the element, as well as that produced by membrane elements located upstream relative to the permeate flow direction for that pressure vessel. The permeate water that is collected within the permeate carrier tube of the vessel's first element flows into the adjacent element's permeate carrier tube through an interconnecting component between the tubes where it combines with the permeate water produced by that element. This combined permeate water will then travel through another interconnecting component into the next element and so forth through the remaining membrane elements within the vessel until the combined permeate water from all of the vessel elements exits the opposite end of the vessel into a permeate collection piping manifold.

One type of interconnecting method between membrane elements, called an interconnector, consists of a hollow cylinder or pipe that uses elastomer O-rings to seal each end of the interconnector against the permeate carrier tubes of adjacent membrane elements. Another type of interconnection method uses a direct facial connection between the ends of adjacent membrane elements that also uses an O-ring to seal and isolate the higher pressure saline water from the lower pressure permeate water within the permeate carrier tubes. Both the first and last membrane element in the pressure vessel will use an interconnecting component to potentially transport the permeate water from the membrane elements to the vessel end caps, although the permeate water may only be removed from one of those two end caps.

The electrical conductance of water, also known as the water conductivity, is commonly measured using a pair of electrodes that are connected to an alternative voltage source, and is roughly proportional to the concentration of dissolved salts in the water. Water conductivity is a convenient way to monitor the dissolved salt concentration in the various RO or NF streams entering or exiting the membrane vessels as a means of monitoring how well the membrane system is separating out dissolved salts from the inlet water into a concentrated stream (aka, concentrate) in producing the low salinity permeate water. Leaking or missing interconnection O-rings or poorly performing membrane elements will allow increased amounts of saline water from the higher pressure side of the membrane to enter the permeate carrier tubes at those compromised locations before combining with permeate water produced by other membrane elements, and so will increase the conductivity of the permeate water exiting the membrane vessel, as well as the conductivity of the combined permeate water from the entire membrane system. Measuring the conductivity of permeate water exiting the individual membrane vessels makes it possible to identify a vessel that is providing permeate water with higher conductivity than desirable and so might contain one or more poorly performing membrane elements or leaking interconnecting components. Once a problematic membrane vessel is identified, the specific location of the problem within the vessel must be isolated before it can be repaired by replacing the responsible membrane elements or by installing new O-rings in leaking interconnecting components.

Fouling of the RO or NF membrane by the deposition of particles from its feed water or by the formation of a salt scale can also increase membrane salt passage into the permeate water. Identifying the specific region where there is high dissolved salt passage relative to other regions within the vessels' membrane elements provides evidence that can be critical in identifying the type of problem that is responsible for an increase in the combined permeate water conductivity. For example, an increase in percent salt passage values in membrane elements located at the concentrate end of the membrane system, where the high pressure water contains the highest saline concentration, would provide evidence of the formation of a salt scale within those membrane elements.

A method that has been commonly used to characterize the relative permeate quality within a vessel's membrane elements and interconnectors is called “probing”. While the membrane system is not operating and under pressure, small diameter tubing, such as one-quarter inch in diameter, is inserted through an otherwise plugged permeate port often located on the pressure vessel's opposite end cap from where the vessel permeate exits into a permeate collection manifold. The tubing is worked through the permeate carrier tubes and interconnecting components of the membrane elements until the tubing reaches the permeate manifold end of the vessel. The tubing is then held or secured in some manner at its point of vessel entry while the system operation is engaged and the saline water is pressurized. While the membrane system is operating, the tubing serves to separate out permeate water from specific sampling locations within the membrane permeate carrier tubes so that the electrical conductance of the water at those isolated locations can be measured externally from the membrane vessel using a portable conductivity instrument. After recording the electrical conductance at a starting location and while the system continues to operate under pressure, the tubing may be withdrawn a particular distance, such as half of the length of a membrane element, so that permeate water isolated from this new location may be tested for its conductivity. This procedure might be repeated until the permeate conductivity is mapped out as a function of linear distance through the permeate carrier tubes and interconnecting components of the entire vessel.

“Probing” can be a difficult procedure to perform safely and accurately. The membrane system permeate water is usually under some amount of pressure, such that the process of “probing” often sprays water around the entry port on the vessel end cap, which may expose equipment to the water, and may contact the personnel involved with controlling the movement of the tubing. The permeate pressure places a force on the “probing” tubing that increases the difficulty in controlling the distance at which the tubing is removed through the vessel, which can result in inaccurate assessment of the problem locations within the vessel. Also, the exiting water flow through the tubing and out the entry port on the end cap will reduce the permeate pressure within the vessel, which then affects the rate of membrane water permeation within that vessel and subsequently the dissolved salt concentration and conductivity of its permeate water. Furthermore, accurate calculation of the percent salt passage values at each permeate location within the vessel would require knowledge of the particular split of the permeate water between the percentage which exits the vessel out through the permeate manifold end, the percentage that escapes out the end cap connection through which the probing tube was inserted, and the remaining percentage that flows through the sample tubing, but measurement of these flow rates is not feasible.

Therefore, a need exists for a device that can accurately and conveniently measure the relative dissolved salt concentration at specific distance intervals throughout the permeate carrier tubes from within a sealed RO or NF membrane vessel so that the permeate water flow rate or path is not altered, and for that device to include a microprocessor to calculate the percent salt passage specific to each permeate sensor location that compensates for the effect of the permeate water quality that originated from other locations, and that incorporates the measured or estimated dissolved salt concentration on the higher pressure side of the membrane that would be associated with each permeate sensor location.

### BRIEF SUMMARY OF THE INVENTION

This invention uses multiple pairs of electrodes acting as electrical conductivity sensors that are located within the spiral wound membrane elements and their interconnecting components installed within a pressure vessel. By locating the sensors within the vessel, the permeate stream can follow its normal direction of flow and so will not affect the rate at which the membrane separates out dissolved salt. The use of multiple conductivity sensors within the permeate carrier tubes and interconnecting components makes it possible to calculate the dissolved salt concentration that would be present at each permeate sensor location as if there was no permeate water flowing into that location from other membrane elements or regions. A percent passage of dissolved salt specific to that location can then be calculated for each permeate sensor reading by comparing this corrected permeate salt concentration to either the dissolved salt concentration derived from a conductivity measurement located on the saline side of the membrane in the same vicinity as the permeate reading, or it can be calculated from an estimated conductivity derived for that location. Identifying inconsistently high percent salt passage values relative to other permeate locations makes it possible to identify where O-rings may be leaking, where a poorly performing membrane element might be located, or where there might be a region of membrane where particle fouling or the formation of a salt scale might be occurring.

### BRIEF DESCRIPTION OF THE SEVERAL VIEWS OF THE DRAWINGS

Some embodiments of the present invention are illustrated as an example and are not limited by the figures of the accompanying drawings, in which like references may indicate similar elements and in which:

FIG. 1 is an illustration of a preferred embodiment of the invention, which has multiple conductivity sensors attached to electrical wires and a support material with a watertight plug for installation in a spiral wound membrane pressure vessel.

FIG. 2 is an illustration of a spiral wound membrane vessel with the preferred embodiment of the invention illustrated in FIG. 1 inserted into the permeate carrier tubes and interconnecting components and secured to a plug threaded into the vessel end cap.

FIG. 3 is an illustration of a spiral wound membrane vessel with a preferred embodiment of the invention that uses independent electrode assemblies that are secured to locations within the membrane elements and interconnecting components.

FIG. 4 shows the bottom part of an enclosure for the printed circuit boards with microprocessor and other associated electronic components, according to various embodiments described herein.

FIG. 5 shows the top part of an enclosure for the printed circuit boards with microprocessor and other associated electronic components, according to various embodiments described herein.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The present disclosure is to be considered as an exemplification of the invention, and is not intended to limit the invention to the specific embodiments illustrated by the fixtures of description below.

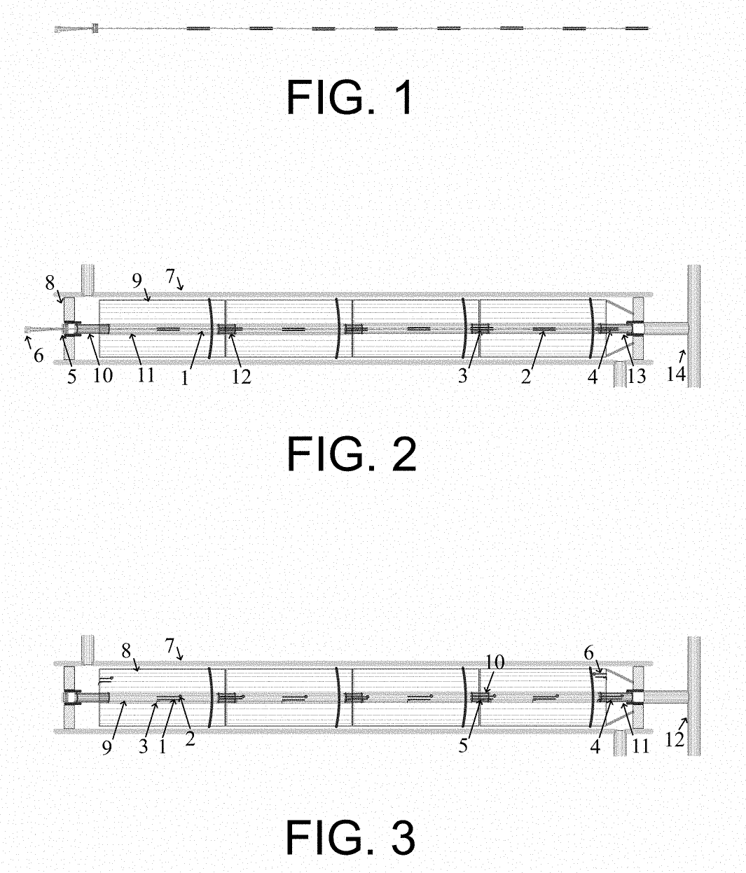

The present invention will now be described by referencing the appended figures representing preferred embodiments. FIG. 1 depicts a preferred embodiment of the invention that uses multiple electrode pairs, such as the identified electrode pair 1, that act as separate conductivity sensors and are attached to electrical wires 2 and to a semi rigid material at specific distance intervals from the other permeate sensor locations. The cord assembly includes a watertight plug 3 and electrical end connections 4, such as RJ45 electrical connectors.

FIG. 2 depicts how the preferred embodiment illustrated in FIG. 1 inserts into a pressure vessel 7 of a reverse osmosis or nanofiltration membrane system, through the vessel end cap 8, through the permeate interconnecting component 10 with the end cap into a permeate carrier tube 11 of a membrane element 9 and proceeds through the interconnecting component 12 with the adjacent membrane element and on through other interconnecting components and membrane elements, and might end within the interconnecting component 13 at the opposite end of the vessel where the permeate collection manifold 14 for that vessel is located. The cord 1 has conductivity sensors specifically positioned such that a sensor might be located within the middle of each membrane element, such as the identified sensor 2, and might be within the interconnecting components between the permeate carriers of adjacent membrane elements, such as the identified interconnecting component 3. A conductivity sensor might also be located within the interconnecting component 4 on the outlet end of the vessel permeate stream before it combines in the permeate collection manifold with the permeate water produced from other membrane vessels. A plug 5 provides a watertight seal with the end cap at its point of insertion to force the entire vessel permeate stream to continue its normal direction of flow to the permeate collection manifold. The electrical wires from the conductivity sensors proceed through the plug to end connectors 6.

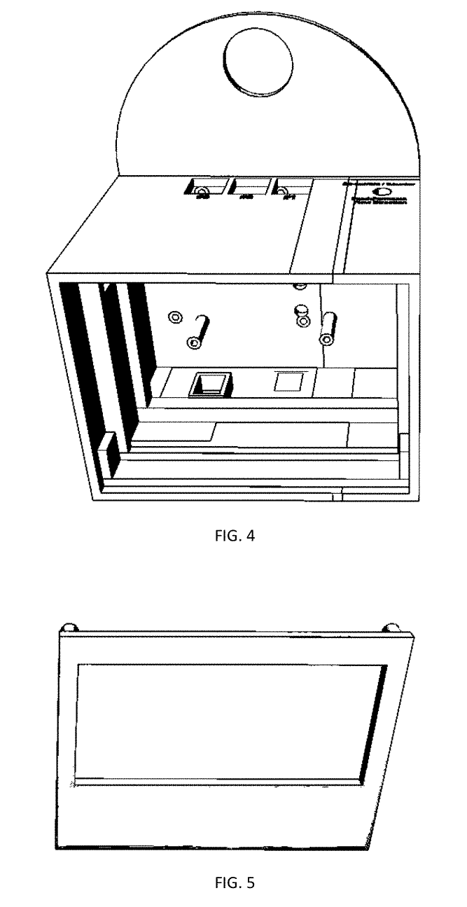

FIG. 3 depicts a preferred embodiment of the invention that uses independent sensor assemblies, each consisting of a pair of electrodes 1 that electrically connect with a wireless transmitter and battery 2 and a securing device 3 that allows the sensor assembly to be attached to the permeate carrier tube 9 of a spiral wound membrane element 8 within its pressure vessel 7 of a reverse osmosis or nanofiltration membrane system. A sensor assembly 5 might also be attached within an interconnecting component 10 located between adjacent membrane elements. A sensor assembly 4 might also be attached within an interconnecting component 11 on the outlet end of the vessel permeate stream before the water combines in the permeate collection manifold 12 with the permeate water produced from other membrane vessels. A sensor assembly 6 might also be attached to the membrane elements at one or more locations within the high pressure saline water of the membrane system in order to measure the salinity in the vicinity of the permeate sensor assemblies for increased accuracy in calculating the percent salt passage values.

FIG. 4 depicts a preferred embodiment of how the base section of an enclosure might be located externally of the membrane vessel for holding the wire connections from the conductivity sensor cord and where printed circuit boards, the microprocessor, and affiliated electronics would be located. The microprocessor evaluates the conductivity readings of the sensors and evaluates each permeate sensor reading relative to the values measured at other permeate sensor locations within the vessel, as well as relative to conductivity readings obtained from conductivity sensors located on the high pressure saline side of the membrane either within the pressure vessel or located externally of the vessel as appropriate for calculating a local percent salt passage value associated with each permeate sensor location.

FIG. 5 depicts a preferred embodiment of how the enclosure top would be designed for a display of the sensor conductivity readings and of the percent salt passage values calculated by the microprocessor, although said microprocessor and display might alternatively be located within the control panel of the membrane system and also used for other monitoring and control functions required for the membrane system.

In accordance with the provisions of the patent statutes, the principle and mode of operation of this invention have been explained and illustrated in its preferred embodiments. However, it must be understood that this invention may be practiced otherwise than as specifically explained and illustrated without departing from its spirit or scope.

What is claimed is:

1. A device comprising multiple pairs of conductivity electrodes wherein each electrode set is secured within a spiral wound membrane vessel.

2. The invention in claim 1 further comprising electrical wires with electrical connectors and a semi rigid support material, wherein said pairs of electrodes are secured in locations within the permeate carrier tubes and interconnecting components of the spiral wound membrane elements by attachment to said support material and to said electrical connection wires.

3. The invention in claim 2 further comprising a water sealing plug, wherein said electrical wires proceed through said plug that creates a watertight seal between the sensor cord and the permeate connection port of one of the end caps of a membrane vessel, but allows the wires to proceed through the plug to end connectors located outside of the pressure vessel.

4. The invention in claim 3 wherein the conductance measurements from multiple locations are evaluated and used by a microprocessor to calculate a value for the percentage of dissolved salts passing through the membrane at the electrode pair locations along the permeate stream path as it flows through the permeate carrier tubes and interconnecting components of the membrane elements within their vessel.

5. The invention in claim 1 further comprising wireless transmitting components, batteries, and physical attachment components, wherein each electrode pair is secured to a membrane element in either permeate stream or high pressure locations via said physical attachment component and is electrically connected to said battery and to said transmitting component to communicate the electrical conductance at each electrode pair through said electronic transmitting components.

6. The invention in claim 5 wherein the conductance measurements from multiple locations are used by a microprocessor to calculate a value for the percentage of dissolved salts passing through the membrane at electrode pair locations along the permeate stream path as it flows through the permeate carrier tubes and interconnecting components of the membrane elements within their vessel.
